(19) United States
(12) Patent Application Publication
(10) Pub. No.: US 2007/0219245 A1
(43) Pub. Date: Sep. 20, 2007

(54) METHOD OF USE FOR SUBSTITUTED DIPIPERIDINE CCR2 ANTAGONISTS

(76) Inventors: Cuifen Hou, Spring House, PA (US); Yin Liang, Ambler, PA (US); Keith T. Demarest, Flemington, NJ (US); Druie E. Cavender, Flemington, NJ (US); Michael P. Wachter, Bloomsbury, NJ (US); Mingde Xia, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/685,247

(22) Filed: Mar. 13, 2007

Related U.S. Application Data
(60) Provisional application No. 60/782,040, filed on Mar. 14, 2006.

(51) Int. Cl. A61K 31/4545

(52) U.S. Cl. 514/316

(57) ABSTRACT

The present invention is directed to a method for use of substituted dipiperidine compounds of Formula (I)

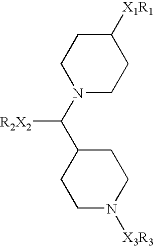

or a salt, isomer, prodrug, metabolite or polymorph thereof, which are CCR2 antagonists, for preventing, treating or ameliorating syndromes, disorders or diseases related to CCR2 activation in a subject in need thereof.

### CROSS REFERENCE TO RELATED APPLICATIONS

This present application claims benefit of U.S. Provisional Patent Application Ser. No. 60/782040, filed Mar. 14, 2006, which is incorporated herein by reference in its entirety and for all purposes.

### FIELD OF THE INVENTION

The invention is directed to a method for use of substituted dipiperidine compounds, which are antagonists to the chemoattractant cytokine receptor 2 (CCR2) and pharmaceutical compositions thereof. More particularly, the CCR2 antagonists are substituted dipiperidine carboxylic acid, alcohol and ester compounds useful in a method for preventing, treating or ameliorating a syndrome, disorder or disease related to CCR2 activation.

### BACKGROUND OF THE INVENTION

CCR2 is a member of the GPCR family of receptors, as are all known chemokine receptors, and are expressed by monocytes and memory T-lymphocytes. The CCR2 signaling cascade involves activation of phospholipases (PLCβ₂), protein kinases (PKC), and lipid kinases (PI-3 kinase).

Chemoattractant cytokines (i.e., chemokines) are relatively small proteins (8-10 kD), which stimulate the migration of cells. The chemokine family is divided into four subfamilies based on the number of amino acid residues between the first and second highly conserved cysteines.

Monocyte chemotactic protein-1 (MCP-1) is a member of the CC chemokine subfamily (wherein CC represents the subfamily having adjacent first and second cysteines) and binds to the cell-surface chemokine receptor 2 (CCR2). MCP-1 is a potent chemotactic factor, which, after binding to CCR2, mediates monocyte and lymphocyte migration (i.e., chemotaxis) toward a site of inflammation. MCP-1 is also expressed by cardiac muscle cells, blood vessel endothelial cells, fibroblasts, chondrocytes, smooth muscle cells, mesangial cells, alveolar cells, T-lymphocytes, marcophages, and the like.

After monocytes enter the inflammatory tissue and differentiate into macrophages, monocyte differentiation provides a secondary source of several proinflammatory modulators, including tumor necrosis factor-α (TNF-α), interleukin-1 (IL-1), IL-8 (a member of the CXC chemokine subfamily, wherein CXC represents one amino acid residue between the first and second cysteines), IL-12, arachidonic acid metabolites (e.g., PGE₂ and LTB₄), oxygen-derived free radicals, matrix metalloproteinases, and complement components.

Animal model studies of chronic inflammatory diseases have demonstrated that inhibition of binding between MCP-1 and CCR2 by an antagonist suppresses the inflammatory response. The interaction between MCP-1 and CCR2 has been implicated (see Rollins B J, Monocyte chemoattractant protein 1: a potential regulator of monocyte recruitment in inflammatory disease, Mol. Med. Today, 1996, 2:198; and Dawson J, et al., Targeting monocyte chemoattractant protein-1 signaling in disease, Expert Opin. Ther. Targets, Feb. 7, 2003 (1):35-48) in inflammatory disease pathologies such as psoriasis, uveitis, atherosclerosis, rheumatoid arthritis, multiple sclerosis, Crohn's Disease, nephritis, organ allograft rejection, fibroid lung, renal insufficiency, Type II diabetes and diabetic complications, diabetic nephropathy, diabetic retinopathy, diabetic retinitis, diabetic microangiopathy, tuberculosis, sarcoidosis, invasive staphylococcia, inflammation after cataract surgery, allergic rhinitis, allergic conjunctivitis, chronic urticaria, Chronic Obstructive Pulmonary Disease (COPD), allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, lung, prostate, and stomach.

Monocyte migration is inhibited by MCP-1 antagonists (either antibodies or soluble, inactive fragments of MCP-1), which have been shown to inhibit the development of arthritis, asthma, and uveitis. Both MCP-1 and CCR2 knockout (KO) mice have demonstrated that monocyte infiltration into inflammatory lesions is significantly decreased. In addition, such KO mice are resistant to the development of experimental allergic encephalomyelitis (EAE, a model of human MS), cockroach allergen-induced asthma, atherosclerosis, and uveitis. Rheumatoid arthritis and Crohn's Disease patients have improved during treatment with TNF-α antagonists (e.g., monoclonal antibodies and soluble receptors) at dose levels correlated with decreases in MCP-1 expression and the number of infiltrating macrophages.

MCP-1 has been implicated in the pathogenesis of seasonal and chronic allergic rhinitis, having been found in the nasal mucosa of most patients with dust mite allergies. MCP-1 has also been found to induce histamine release from basophils in vitro. During allergic conditions, both allergens and histamines have been shown to trigger (i.e., to up-regulate) the expression of MCP-1 and other chemokines in the nasal mucosa of people with allergic rhinitis, suggesting the presence of a positive feedback loop in such patients.

CCR2 influences the development of obesity and associated adipose tissue inflammation and systemic insulin resistance and plays a role in the maintenance of adipose tissue macrophages and insulin resistance once obesity and its metabolic consequences are established (J. Clin. Invest., 2006, 116, 115-124).

There remains a need for a method for preventing, treating or ameliorating a syndrome, disorder or disease related to CCR2 activation.

All documents cited herein are incorporated by reference.

### SUMMARY OF THE INVENTION

The invention provides a method for use of substituted dipiperidine compounds of Formula (I) or a salt, isomer, prodrug, metabolite or polymorph thereof, for preventing, treating or ameliorating a syndrome, disorder or disease related to CCR2 activation in a subject in need thereof:

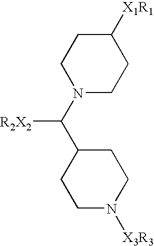

which are CCR2 antagonists.

United States Patent Publication US20060069123 and PCT Application Publication WO2006036527 describe compounds of Formula (I) intended to be encompassed for use in the method of the present invention, which Publications are incorporated herein by reference in their entirety and for all purposes.

The present invention also provides a method for preventing, treating or ameliorating a syndrome, disorder or disease related to CCR2 activation in a subject in need thereof comprising administering to the subject an effective amount of a compound of Formula (I) or a form, composition or medicament thereof.

### DETAILED DESCRIPTION OF THE INVENTION

The present invention is directed to a method for use of a compound of Formula (I) or a salt, isomer, prodrug, metabolite or polymorph thereof for preventing, treating or ameliorating a syndrome, disorder or disease related to CCR2 activation in a subject in need thereof:

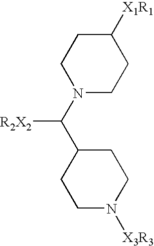

wherein
X₁ is absent, alkyl, carbonyl, alkylcarbamoyl or alkylcarbamoylalkyl,
R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, cyano, halogen, hydroxy, hydroxyalkyl, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, alkylamino, alkylaminoalkyl, sulfonylamino, sulfonylaminoalkyl, alkylsulfonylamino, alkylsulfonylaminoalkyl, carboxy, acyl, carbonylalkoxy, carbamoyl or carbamoylalkyl,
X₂ is absent or alkyl,
R₂ is hydroxy, halogen, amino (optionally substituted with one or more of alkyl, formyl, acyl, sulfonylalkyl or carbonylalkoxy), cyano, nitro, alkoxy, carboxy, carbonylalkoxy, oxyacyl, oxyacylaryl, oxyacrylyl, oxyacrylylaryl (optionally substituted on aryl with one or more of alkyl, alkoxy, cyano, halogen, hydroxy, nitro, amino or aminoalkyl), oxycarbonylalkoxy, aminoacylamino, aminoacylaminoalkyl, carbamoyl, carbamoylalkyl, urea or ureaalkyl,
X₃ is carbonyl, carboxyl, acyl, acyloxy, acrylyl, carbonylalkynyl, carbonylalkoxy, carbamoyl, carbamoylalkyl, alkylcarbamoyl, thiocarbamyl or iminomethylaminocarbonyl, wherein when X₃ is carbonylalkoxy, then R₃ is optionally present, and
R₃ is cycloalkyl, aryl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or aryl (optionally substituted on aryl with one or more of alkyl, alkoxy, halogen, hydroxy, nitro, amino or aminoalkyl).

An example of the present invention is a method for use of compounds of Formula (I) described in United States Patent Publication US20060069123 and PCT Application Publication WO2006036527 or a salt, isomer, prodrug, metabolite or polymorph thereof for preventing, treating or ameliorating a syndrome, disorder or disease related to CCR2 activation in a subject in need thereof.

An example of the present invention is a method for use of (S)-{[4-(1H-indol-3-yl)-piperidin-1-yl ]}-{1-[(2E)-3-(3,4,5-triflouro-phenyl)-acroloyl]-piperidin-4-yl}-acetic acid or a salt, isomer, prodrug, metabolite or polymorph thereof for preventing, treating or ameliorating a syndrome, disorder or disease related to CCR2 activation in a subject in need thereof.

### Chemical Definitions

As used herein, the following terms have the following meanings.

The term “alkyl” means a saturated aliphatic branched or straight-chain monovalent hydrocarbon radical or linking group substituent having from 1-8 carbon atoms, wherein the radical is derived by the removal of one hydrogen atom from a carbon atom and the linking group is derived by the removal of one hydrogen atom from each of two carbon atoms in the chain. The term includes, without limitation, methyl, methylene, ethyl, ethylene, propyl, propylene, isopropyl, isopropylene, n-butyl, n-butylene, t-butyl, t-butylene, pentyl, pentylene, hexyl, hexylene and the like. An alkyl substituent may be attached to a core molecule via a terminal carbon atom or via a carbon atom within the chain. Similarly, any number of substituent variables may be attached to an alkyl substituent when allowed by available valences. The term “lower alkyl” means an alkyl substituent having from 1-4 carbon atoms.

The term “alkenyl” means a partially unsaturated alkyl radical or linking group substituent having at least at least two carbon atoms and one double bond derived by the removal of one hydrogen atom from each of two adjacent carbon atoms in the chain. Atoms may be oriented about the double bond in either the cis (E) or trans (Z) conformation. The term includes, without limitation, methylidene, vinyl, vinylidene, allyl, allylidene, propylidene, isopropenyl, iso-propylidene, prenyl, prenylene (3-methyl-2-butenylene), methallyl, methallylene, allylidene (2-propenylidene), crotylene (2-butenylene), and the like. An alkenyl substituent may be attached to a core molecule via a terminal carbon atom or via a carbon atom within the chain. Similarly, any number of substituent variables may be attached to an alkenyl substituent when allowed by available valences. The term “lower alkenyl” means an alkenyl substituent having from 2-4 carbon atoms.

The term “alkynyl” means a partially unsaturated alkyl radical or linking group substituent having at least two carbon atoms and one triple bond derived by the removal of two hydrogen atom from each of two adjacent carbon atoms in the chain. The term includes, without limitation, ethinyl, ethinylidene, propargyl, propargylidene and the like. An alkynyl substituent may be attached to a core molecule via a terminal carbon atom or via a carbon atom within the chain. Similarly, any number of substituent variables may be attached to an alkynyl substituent when allowed by available valences. The term “lower alkynyl” means an alkynyl substituent having from 2-4 carbon atoms.

The term “alkoxy” means an alkyl radical or linking group substituent attached through an oxygen-linking atom, wherein a radical is of the formula —O-alkyl and a linking group is of the formula —O-alkyl-. The term includes, without limitation, methoxy, ethoxy, propoxy, butoxy and the like. An alkoxy substituent may be attached to a core molecule and further substituted where allowed.

The term “cycloalkyl” means a saturated or partially unsaturated monocyclic, polycyclic or bridged hydrocarbon ring system radical or linking group. A ring of 3 to 20 carbon atoms may be designated by C₃₋₂₀ cycloalkyl; a ring of 3 to 12 carbon atoms may be designated by C₃₋₁₂ cycloalkyl, a ring of 3 to 8 carbon atoms may be designated by C₃₋₈ cycloalkyl and the like.

The term cycloalkyl includes, without limitation, cyclopropyl, cyclobutyl, cyclopentyl, cyclohexyl, cyclohexenyl, cycloheptyl, cyclooctyl, indanyl, indenyl, 1,2,3,4-tetrahydro-naphthalenyl, 5,6,7,8-tetrahydro-naphthalenyl, 6,7,8,9-tetrahydro-5H-benzocycloheptenyl, 5,6,7,8,9,10-hexahydro-benzocyclooctenyl, fluorenyl, bicyclo[2.2.1]heptyl, bicyclo[2.2.1]heptenyl, bicyclo[2.2.2]octyl, bicyclo[3.1.1]heptyl, bicyclo[3.2.1]octyl, bicyclo[2.2.2]octenyl, bicyclo[3.2.1]octenyl, adamantanyl, octahydro-4,7-methano-1H-indenyl, octahydro-2,5-methano-pentalenyl (also referred to as hexahydro-2,5-methano-pentalenyl) and the like. A cycloalkyl substituent may be attached to a core molecule and further substituted where allowed.

The term “aryl” means an unsaturated, conjugated π electron monocyclic or polycyclic hydrocarbon ring system radical or linking group substituent of 6, 9, 10 or 14 carbon atoms. The term includes, without limitation, phenyl, naphthalenyl, fluorenyl, indenyl, azulenyl, anthracenyl and the like. An aryl substituent may be attached to a core molecule and further substituted where allowed.

The term “heterocyclyl” means a saturated, partially unsaturated (such as those named with the prefix dihydro, trihydro, tetrahydro, hexahydro and the like) or unsaturated monocyclic, polycyclic or bridged hydrocarbon ring system radical or linking group substituent, wherein at least one ring carbon atom has been replaced with one or more heteroatoms independently selected from N, O or S. A heterocyclyl substituent further includes a ring system having up to 4 nitrogen atom ring members or a ring system having from 0 to 3 nitrogen atom ring members and 1 oxygen or sulfur atom ring member. Alternatively, up to two adjacent ring members may be a heteroatom, wherein one heteroatom is nitrogen and the other is selected from N, O or S. A heterocyclyl radical is derived by the removal of one hydrogen atom from a single carbon or nitrogen ring atom. A heterocyclyl linking group is derived by the removal of one hydrogen atom from two of either a carbon or nitrogen ring atom. A heterocyclyl substituent may be attached to a core molecule by either a carbon atom ring member or by a nitrogen atom ring member and further substituted where allowed.

The term heterocyclyl includes, without limitation, furanyl, thienyl, 2H-pyrrolyl, 2-pyrrolinyl, 3-pyrrolinyl, pyrrolidinyl, pyrrolyl, 1,3-dioxolanyl, oxazolyl, thiazolyl, imidazolyl, 2-imidazolinyl (also referred to as 4,5-dihydro-1H-imidazolyl), imidazolidinyl, 2-pyrazolinyl, pyrazolidinyl, pyrazolyl, isoxazolyl, isothiazolyl, oxadiazolyl, triazolyl, thiadiazolyl, tetrazolyl, tetrazolinyl, tetrazolidinyl, 2H-pyranyl, 4H-pyranyl, thiopyranyl, pyridinyl, piperidinyl, 1,4-dioxanyl, morpholinyl, 1,4-dithianyl, thiomorpholinyl, pyridazinyl, pyrimidinyl, pyrazinyl, piperazinyl, azetidinyl, azepanyl, indolizinyl, indolyl, 4-aza-indolyl (also referred to as 1H-pyrrolo[3,2-b]pyridin-3-yl), 6-aza-indolyl (also referred to as 1H-pyrrolo[2,3-c]pyridin-3-yl), 7-aza-indolyl (also referred to as 1H-pyrrolo[2,3-b]pyridin-3-yl), isoindolyl, 3H-indolyl, indolinyl, benzo[b]furanyl, furo[2,3-b]pyridin-3-yl, benzo[b]thienyl, indazolyl (also referred to as 1H-indazolyl), benzoimidazolyl, benzothiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl, quinuclidinyl, 2H-chromenyl, 3H-benzo[f]chromenyl, tetrahydro-furanyl, tetrahydro-thienyl, tetrahydro-pyranyl, tetrahydro-thiopyranyl, tetrahydro-pyridazinyl, hexahydro-1,4-diazepinyl, hexahydro-1,4-oxazepanyl, 2,3-dihydro-benzo[b]oxepinyl, 1,3-benzodioxolyl (also known as 1,3-methylenedioxyphenyl or benzo[1,3]dioxolyl), 2,3-dihydro-1,4-benzodioxinyl (also known as 1,4-ethylenedioxyphenyl or benzo[1,4]dioxinyl), benzo-dihydro-furanyl (also known as 2,3-dihydro-benzofuranyl), benzo-tetrahydro-pyranyl, benzo-dihydro-thienyl, 5,6,7,8-tetrahydro-4H-cyclohepta[b]thienyl, 5,6,7-trihydro-4H-cyclohexa[b]thienyl, 5,6-dihydro-4H-cyclopenta[b]thienyl, 2-aza-bicyclo[2.2.1]heptyl, 1-aza-bicyclo[2.2.2]octyl, 8-aza-bicyclo[3.2.1]octyl, 7-oxa-bicyclo[2.2.1]heptyl, pyrrolidinium, piperidinium, piperazinium, morpholinium and the like.

The term “acrylyl” means a linking group of the formula —C(O)C═C—.

The term “acyl” means a radical of the formula —C(O)-alkyl, or a linking group of the formula —C(O)-alkyl-.

The term “acyloxy” means a linking group of the formula —C(O)-alkyl-O—.

The term “alkoxycarbonylalkoxy” means a radical of the formula —O-alkyl-C(O)O-alkyl, or a linking group of the formula —O-alkyl-C(O)O-alkyl-.

The term “alkoxycarboxy” means a radical of the formula —O-alkyl-CO₂H or —O-alkyl-C(O)OH.

The term “alkylamino” means a radical of the formula -alkyl-NH₂, or a linking group of the formula -alkyl-NH—.

The term “alkylaminoalkyl” means a radical of the formula -alkyl-NH-alkyl or -alkyl-N(alkyl)₂, or a linking group of the formula -alkyl-NH-alkyl- or -alkyl-N(alkyl)-alkyl-.

The term “alkylcarbamoyl” means a radical of the formula -alkyl-C(O)NH₂, or a linking group of the formula -alkyl-C(O)NH—.

The term “alkylcarbamoylalkyl” means a radical of the formula -alkyl-C(O)NH-alkyl or -alkyl-C(O)N(alkyl)₂, or a linking group of the formula -alkyl-C(O)NH-alkyl- or —C(O)N(alkyl)-alkyl-.

The term “alkylcarbonylalkoxy” means a radical of the formula -alkyl-C(O)O-alkyl, or a linking group of the formula -alkyl-C(O)O-alkyl-.

The term “alkylcarboxy” means a radical of the formula -alkyl-CO₂H or -alkyl-C(O)OH.

The term “alkylsulfonylamino” means a radical of the formula -alkyl-SO₂—NH₂.

The term “alkylsulfonylaminoalkyl” means a radical of the formula -alkyl-SO₂—NH-alkyl or -alkyl-SO₂—N(alkyl)₂, or a linking group of the formula -alkyl-SO₂—NH-alkyl- or -alkyl-SO₂—N(alkyl)-alkyl-.

The term “amino” means a radical of the formula —NH₂.

The term “aminoacylamino” means a radical of the formula —NH—C(O)-alkyl-NH₂, or a linking group of the formula —NH—C(O)-alkyl-NH—.

The term “aminoacylaminoalkyl” means a radical of the formula —NH—C(O)-alkyl-NH-alkyl or —NH—C(O)-alkyl-N(alkyl)₂, or a linking group of the formula —NH—C(O)-alkyl-NH-alkyl- or —NH—C(O)-alkyl-N(alkyl)-alkyl-.

The term “aminoalkyl” means a radical of the formula —NH-alkyl or —N(alkyl)₂, or a linking group of the formula —NH-alkyl- or —N(alkyl)-alkyl-.

The term “carbamoyl” means a radical of the formula —C(O)NH₂, or a linking group of the formula —C(O)NH—.

The term “carbamoylalkyl” means a radical of the formula —C(O)NH-alkyl or —C(O)N(alkyl)₂, or a linking group of the formula —C(O)NH-alkyl- or —C(O)N(alkyl)-alkyl-.

The term “carbonyl” means a linking group of the formula —C(O)— or —C(═O)—.

The term “carbonylalkoxy” means a radical of the formula —C(O)O-alkyl, or a linking group of the formula —C(O)O-alkyl-.

The term “carboxy” means a radical of the formula —C(O)OH or —CO₂H.

The term “carboxyl” means a linking group of the formula —C(O)O—.

The term “halo” or “halogen” means fluoro, chloro, bromo or iodo.

The term “iminomethylaminocarbonyl” means a linking group having the formula —C(NH)NHC(O)— or —C(═NH)NHC(O)—.

The term “oxyacyl” means a radical of the formula —OC(O)-alkyl, or a linking group of the formula —OC(O)-alkyl-.

The term “oxyacylaryl” means a radical of the formula —OC(O)-alkyl-aryl.

The term “oxyacrylyl” means a radical of the formula —OC(O)-alkenyl, or a linking group of the formula —OC(O)-alkenyl-.

The term “oxyacrylylaryl” means a radical of the formula —OC(O)-alkenyl-aryl.

The term “oxycarbonylalkoxy” means a radical of the formula —OC(O)—O-alkyl, or a linking group of the formula —OC(O)—O-alkyl-.

The term “sulfonylalkyl” means a radical of the formula —SO₂-alkyl, or a linking group of the formula —SO₂-alkyl-.

The term “sulfonylamino” means a radical of the formula —SO₂—NH₂.

The term “sulfonylaminoalkyl” means a radical of the formula —SO₂—NH-alkyl or —SO₂—N(alkyl)₂, or a linking group of the formula —SO₂—NH-alkyl- or —SO₂—N(alkyl)-alkyl-.

The term “thioalkyl” means a radical of the formula —S-alkyl, or a linking group of the formula —S-alkyl-.

The term “thiocarbamyl” means a radical of the formula —C(S)NH₂ or —C(═S)NH₂, or a linking group of the formula —C(S)NH—.

The term “urea” means a radical of the formula —NH—C(O)—NH₂.

The term “ureaalkyl” means a radical of the formula —NH—C(O)—NH-alkyl or —NH—C(O)—N(alkyl)₂.

The term “substituted” means one or more hydrogen atoms on a core molecule have been replaced with one or more radicals or linking groups, wherein the linking group, by definition is also further substituted.

The term “dependently selected” means one or more substituent variables are present in a specified combination (e.g. groups of substituents commonly appearing in a tabular list).

The substituent nomenclature used in the disclosure of the present invention was derived using nomenclature rules well known to those skilled in the art (e.g., IUPAC).

### Compound Forms

The compounds of the invention may be present in a form which may, alternatively or in addition to a compound of Formula (I), comprise a salt of a compound of Formula (I) or a prodrug or active metabolite of such a compound or salt.

The compounds of the invention may be present in a salt form. For use in medicines, the salts of the compounds of this invention refer to non-toxic “pharmaceutically acceptable salts.” FDA-approved pharmaceutically acceptable salt forms include pharmaceutically acceptable acidic/anionic or basic/cationic salts.

Pharmaceutically acceptable acidic/anionic salts include, without limitation, acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, triethiodide trifluoroacetate salts and the like.

Organic or inorganic acids also include, and are not limited to, hydroiodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic, trifluoroacetic acid and the like.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine (TEA), zinc and the like.

The compounds of the invention may be present in the form of pharmaceutically acceptable prodrugs and metabolites thereof. In general, such prodrugs and metabolites will be functional derivatives of the compounds that are readily convertible in vivo into an active compound.

The term “prodrug” means a pharmaceutically acceptable form of a functional derivative of a compound of the invention (or a salt thereof), wherein the prodrug may be: 1) a relatively active precursor which converts in vivo to an active prodrug component; 2) a relatively inactive precursor which converts in vivo to an active prodrug component; or 3) a relatively less active component of the compound that contributes to therapeutic biological activity after becoming available in vivo (i.e., as a metabolite). Conventional procedures for the selection and preparation of suitable prodrug derivatives are described in, for example, “Design of Prodrugs”, ed. H. Bundgaard, Elsevier, 1985.

The term “metabolite” means a pharmaceutically acceptable form of a metabolic derivative of a compound of the invention (or a salt thereof), wherein the derivative is a relatively less active component of the compound that contributes to therapeutic biological activity after becoming available in vivo.

The present invention also contemplates compounds of Formula (I) in various stereoisomeric or tautomeric forms. The invention encompasses all such CCR2 inhibiting compounds, including active compounds in the form of essentially pure enantiomers, racemic mixtures and tautomers or pharmaceutically acceptable forms thereof.

The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers).

The term “stereoisomer” refers to isomers of identical constitution that differ in the arrangement of their atoms in space. Enantiomers and diastereomers are stereoisomers wherein an asymmetrically substituted carbon atom acts as a chiral center. The term “chiral” refers to a molecule that is not superposable on its mirror image, implying the absence of an axis and a plane or center of symmetry. The term “enantiomer” refers to one of a pair of molecular species that are mirror images of each other and are not superposable. The term “diastereomer” refers to stereoisomers that are not related as mirror images. The symbols “R” and “S” represent the configuration of substituents around a chiral carbon atom(s). The symbols “R *” and “S*” denote the relative configurations of substituents around a chiral carbon atom(s).

The term “racemate” or “racemic mixture” refers to a compound of equimolar quantities of two enantiomeric species, wherein the compound is devoid of optical activity. The term “optical activity” refers to the degree to which a chiral molecule or nonracemic mixture of chiral molecules rotates the plane of polarized light.

The term “geometric isomer” refers to isomers that differ in the orientation of substituent atoms in relationship to a carbon-carbon double bond, to a cycloalkyl ring or to a bridged bicyclic system. Substituent atoms (other than H) on each side of a carbon-carbon double bond may be in an E or Z configuration. In the “E” configuration, the substituents are on opposite sides in relationship to the carbon-carbon double bond; in the “Z” configuration, the substituents are oriented on the same side in relationship to the carbon-carbon double bond.

Substituent atoms (other than H) attached to a hydrocarbon ring may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”. Substituent atoms (other than H) attached to a bridged bicyclic system may be in an “endo” or “exo” configuration. In the “endo” configuration, the substituents attached to a bridge (not a bridgehead) point toward the larger of the two remaining bridges; in the “exo” configuration, the substituents attached to a bridge point toward the smaller of the two remaining bridges.

It is to be understood that the various substituent stereoisomers, geometric isomers and mixtures thereof used to prepare compounds of the present invention are either commercially available, can be prepared synthetically from commercially available starting materials or can be prepared as isomeric mixtures and then obtained as resolved isomers using techniques well-known to those of ordinary skill in the art.

The isomeric descriptors “R, ” “S” “S*,” “R*,” “E,” “Z” “cis,” “trans,” “exo”, and “endo”, where used herein, indicate atom configurations relative to a core molecule and are intended to be used as defined in the literature.

The compounds of the present invention may be prepared as individual isomers by either isomer-specific synthesis or resolved from an isomeric mixture. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using various well known chromatographic methods.

Furthermore, compounds of the present invention may have a plurality of polymorph or amorphous crystalline forms and, as such, are intended to be included in the scope of the invention. In addition, some of the compounds may form a plurality of solvates with water (i.e., hydrates) or common organic solvents, such are also intended to be encompassed within the scope of this invention.

During any of the processes for preparation of the compounds of the present invention, it may be necessary and/or desirable to protect sensitive or reactive groups on any of the molecules concemed. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

### Therapeutic Use

Compounds of Formula (I) or a form, composition or medicament thereof in accordance with the present invention are CCR2 antagonists and are useful in a method for preventing, treating or ameliorating a syndrome, disorder or disease related to CCR2 activation in a subject in need thereof comprising the step of administering to the subject an effective amount of a compound of Formula (I) or a form, composition or medicament thereof. Accordingly, the present invention is directed to a method for preventing, treating or ameliorating a syndrome, disorder or disease related to CCR2 activation in a subject in need thereof comprising the step of administering to the subject an effective amount of a compound of Formula (I) or form, composition or medicament thereof.

The present invention is also directed to a method for preventing, treating or ameliorating a syndrome, disorder or disease related to CCR2 activation in a subject in need thereof comprising administering to the subject an effective amount of (S)-{[4-(1H-indol-3-yl)-piperidin-1-yl]}-{1-[(2E)-3-(3,4,5-triflouro-phenyl)-acryloyl]piperidin-4-yl}acetic acid or a form, composition or medicament thereof.

The present invention is further directed to a method for preventing, treating or ameliorating Type II diabetes, diabetic complications, diabetic nephropathy, diabetic retinopathy, diabetic retinitis, diabetic microangiopathy and obesity in a subject in need thereof comprising administering to the subject an effective amount of a compound of Formula (I) or a form, composition or medicament thereof.

The present invention is further directed to a method for preventing, treating or ameliorating Type II diabetes, diabetic complications, diabetic nephropathy, diabetic retinopathy, diabetic retinitis, diabetic microangiopathy and obesity in a subject in need thereof comprising administering to the subject an effective amount of (S)-{[4-(1H-indol-3-yl)-piperidin-1-yl]}-{1-[(2E)-3-(3,4,5-triflouro-phenyl)-acryloyl]-piperidin-4-yl}acetic acid or a form, composition or medicament thereof.

The term “administering” with respect to the methods of the invention, means a method for therapeutically or prophylactically preventing, treating or ameliorating a syndrome, disorder or disease as described herein by using a compound of Formula (I) or a form, composition or medicament thereof. Such methods include administering an effective amount of said compound, compound form, composition or medicament at different times during the course of a therapy or concurrently in a combination form. The methods of the invention are to be understood as embracing all known therapeutic treatment regimens.

The term “subject” refers to a patient, which may be animal, typically a mammal, typically a human, which has been the object of treatment, observation or experiment and is at risk of (or susceptible to) developing a syndrome, disorder or disease that is associated with elevated MCP-1 expression or MCP-1 overexpression, or a patient with an inflammatory condition that accompanies syndromes, disorders or diseases associated with elevated MCP-1 expression or MCP-1 overexpression.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

The effective amount of a compound of the invention in such a therapeutic method is from about 0. 1 ng/kg/day to about 300 mg/kg/day.

The invention includes the use of a compound of Formula (I), or a form thereof, for the preparation of a composition or medicament for preventing, treating or ameliorating a syndrome, disorder or disease related to CCR2 activation in a subject in need thereof, wherein the composition or medicament comprises a mixture one or more compounds of the invention and an optional pharmaceutically acceptable carrier.

The term “composition” means a product comprising at least a compound of the invention, such as a product comprising the specified ingredients in the specified amounts, as well as any product which results, directly or indirectly, from such combinations of the specified ingredients in the specified amounts and one or more pharmaceutically acceptable carriers or any such alternatives to a compound of the invention and a pharmaceutically acceptable carrier therefor.

The term “medicament” means a product for use in preventing, treating or ameliorating a syndrome, disorder or disease related to CCR2 activation.

The term “pharmaceutically acceptable” means molecular entities and compositions that are of sufficient purity and quality for use in the formulation of a composition or medicament of the invention and that, when appropriately administered to an animal or a human, do not produce an adverse, allergic, or other untoward reaction. Since both human and veterinary use is included within the scope of the invention, a pharmaceutically acceptable formulation includes a compound of Formula (I) or a form, composition or medicament thereof for either human or veterinary use.

The term “a syndrome, disorder or disease related to CCR2 activation” means, without limitation, syndromes, disorders or diseases associated with elevated MCP-1 expression, MCP-1 overexpression or inflammatory conditions that accompany syndromes, disorders or diseases associated with elevated MCP-1 expression or MCP-1 overexpression.

The terms “elevated MCP-1 expression” or “MCP-1 overexpression” mean unregulated or upregulated CCR2 activation as a result of MCP-1 binding.

The term “unregulated” means unwanted CCR2 activation in a multicellular organism resulting in harm (such as discomfort or decreased life expectancy) to the multicellular organism.

The term “upregulated” means: 1). increased or unregulated CCR2 activity or expression, or 2). increased CCR2 expression leading to unwanted monocyte and lymphocyte migration. The existence of an inappropriate or abnormal level of MCP-1 or activity of CCR2 is determined by procedures well known in the art.

Syndromes, disorders or diseases related to CCR2 activation include, without limitation, ophthalmic disorders, uveitis, atherosclerosis, rheumatoid arthritis, psoriasis, psoriatic arthritis, atopic dermatitis, multiple sclerosis, Crohn's Disease, ulcerative colitis, nephritis, organ allograft rejection, fibroid lung, renal insufficiency, Type II diabetes and diabetic complications, diabetic nephropathy, diabetic retinopathy, diabetic retinitis, diabetic microangiopathy, obesity, tuberculosis, chronic obstructive pulmonary disease, sarcoidosis, invasive staphyloccocia, inflammation after cataract surgery, allergic rhinitis, allergic conjunctivitis, chronic urticaria, asthma, allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, lung, prostate, or stomach.

An example of the invention is a method for preventing, treating or ameliorating a syndrome, disorder or disease related to CCR2 activation selected from Type II diabetes and diabetic complications, diabetic nephropathy, diabetic retinopathy, diabetic retinitis, diabetic microangiopathy and obesity in a subject in need thereof comprising administering to the subject an effective amount of a compound of Formula (I) or a form, composition or medicament thereof.

An example of the invention includes use of the compound of Formula (I) or a form thereof for the manufacture of a medicament for preventing, treating or ameliorating obesity related to CCR2 activation.

An example of the invention includes a method for preventing, treating or ameliorating obesity related to CCR2 activation in a subject in need thereof comprising administering to the subject an effective amount of the compound of Formula (I) or a form thereof, wherein the compound prevents, treats or ameliorates obesity in the subject by reducing weight gain and body fat mass gain.

An effective amount of the compound for use in such a method is in a range of from about 0.001 mg/kg to about 300 mg/kg of body weight per day. Such a method may further comprise administering to the subject an effective amount of the compound as a pharmaceutical composition, medicine or medicament thereof.

An example of the invention includes use of the compound of Formula (I) or a form thereof for the manufacture of a medicament for preventing, treating or ameliorating Type II diabetes and associated diabetic complications related to CCR2 activation.

An example of the invention includes a method for preventing, treating or ameliorating Type II diabetes and associated diabetic complications related to CCR2 activation in a subject in need thereof comprising administering to the subject an effective amount of the compound of Formula (I) or a form thereof, wherein the compound prevents, treats or ameliorates Type II diabetes and associated diabetic complications in the subject by increasing insulin sensitivity.

An effective amount of the compound for use in such a method is in a range of from about 0.001 mg/kg to about 300 mg/kg of body weight per day. Such a method may further comprise administering to the subject an effective amount of the compound as a pharmaceutical composition, medicine or medicament thereof.

The invention includes a method for preventing, treating or ameliorating a syndrome, disorder or disease related to CCR2 activation in a subject in need thereof comprising administering to the subject an effective amount of a compound of Formula (I) or a form, composition or medicament thereof in a combination product with one or more therapeutic agents.

The term “combination product” refers to a compound of Formula (I) or a form, composition or medicament thereof in admixture with a therapeutic agent and an optional carrier for preventing, treating or ameliorating a syndrome, disorder or disease related to CCR2 activation.

The term “therapeutic agent” refers to one or more anti-inflammatory agents (such as a small molecule, antibiotic, corticosteroid, steroid, and the like), anti-infective agents or immunosuppressive agents.

For preventing, treating or ameliorating a syndrome, disorder or disease related to CCR2 activation using a compound of Formula (I) or a form, composition or medicament thereof and a therapeutic agent in a combination product includes, without limitation, co-administration of the compound and the agent, sequential administration of the compound and the agent, administration of a composition containing of the compound and the agent or simultaneous administration of separate compositions containing of the compound and the agent.

As those skilled in the art will appreciate, the effective amounts of the components comprising the combination product may be independently optimized and combined to achieve a synergistic result whereby the pathology is reduced more than it would be if the components of the combination product were used alone.

### Pharmaceutical Compositions

The present invention includes a pharmaceutical composition or medicament comprising one or more of the instant compounds and an optional pharmaceutically acceptable carrier.

The present invention further includes a process for making a pharmaceutical composition or medicament comprising mixing one or more of the instant compounds and an optional pharmaceutically acceptable carrier; and, includes those compositions or medicaments resulting from such a process. Contemplated processes include both conventional and unconventional pharmaceutical techniques.

The composition or medicament may take a wide variety of forms to effectuate mode of administration ocularly, intranasally (by inhalation or insufflation), sublingually, orally, parenterally or rectally including, without limitation, ocular (via a delivery device such as a contact lens and the like), intranasal (via a delivery device), transdermal, topical with or without occlusion, intravenous (both bolus and infusion), injection (intraperitoneally, subcutaneously, intramuscularly, intratumorally, or parenterally) and the like.

The composition or medicament may be in a dosage unit such as a tablet, pill, capsule, powder, granule, liposome, biodegradable carrier, ion exchange resin, sterile solution and the like (facilitating immediate release, timed release, or sustained release), parenteral solution or suspension, metered aerosol or liquid spray, drop, ampoule, auto-injector device or suppository.

Compositions or medicaments suitable for oral administration include solid forms such as pills, tablets, caplets, capsules (each including immediate release, timed release, and sustained release formulations), granules and powders and liquid forms such as solutions, syrups, elixirs, emulsions and suspensions. Forms useful for nasal administration include sterile solutions or nasal delivery devices. Forms useful for ocular administration include sterile solutions or ocular delivery devices. Forms useful for parenteral administration include sterile solutions, emulsions and suspensions.

Alternatively, the composition or medicament may be administered in a form suitable for once-weekly or once-monthly administration. For example, an insoluble salt of the active compound may be adapted to provide a depot preparation for intramuscular injection (e.g., a salt form) or to provide a solution for nasal or ocular administration (e.g., a quaternary ammonium salt).

The dosage form (tablet, capsule, powder, solution, contact lens, patch, liposome, ion exchange resin, suppository, teaspoonful, and the like) containing the composition or medicament thereof contains an effective amount of the active ingredient necessary to provide a therapeutic effect.

The composition or medicament may contain an effective amount of from about 0.0001 mg to about 5000 mg (preferably, from about 0.0001 to about 500 mg) of a compound of the present invention or a pharmaceutically acceptable form thereof and may be constituted into any form suitable for the mode of administration selected for a subject in need.

A contemplated range of the effective amount includes from about 0.0001 mg to about 300 mg/kg of body weight per day. A contemplated range also includes from about 0.0003 to about 100 mg/kg of body weight per day. Another contemplated range includes from about 0.0005 to about 15 mg/kg of body weight per day. The composition or medicament may be administered according to a dosage regimen of from about 1 to about 5 times per day.

For oral administration, the composition or medicament is preferably in the form of a tablet containing, e.g., 0.001, 0.005, 0.01, 0.05, 0.1, 0.5, 1.0, 2.5, 5.0, 10.0, 15.0, 25.0, 50.0, 100, 150, 200, 250, and 500 milligrams of the active ingredient for the symptomatic adjustment of the dosage to the patient to be treated.

Optimal dosages to be administered may be readily determined by those skilled in the art, and will vary with the particular compound used, the mode of administration, the strength of the preparation and the advancement of the disease condition. In addition, factors associated with the particular patient being treated, including patient's sex, age, weight, diet, time of administration and concomitant diseases, will result in the need to adjust dosages. The use of either daily administration or post-periodic dosing may be employed.

### Biological Activity

### EXAMPLE 1

### Diet Induced Obesity Model

In a diet induced obesity (DIO) model in mice, two treatment arms of mice were tested. In the first treatment arm (TA1), DIO mice in four treatment groups (treated: 25 mg/kg, 50 mg/kg and 100 mg/kg and non-treated vehicle control) were fed a high fat diet. In the second treatment arm (TA2), lean mice in two treatment groups (treated: 100 mg/kg and non-treated vehicle control) were fed a regular chow diet.

After 7 days acclimation to the diet, the treated groups in each arm were dosed (0.1 mL, ip, bid) with a test compound from Day 1 to Day 28 of the study. After 7 days acclimation to the diet, the non-treated groups in each arm were dosed (0.1 mL, ip, bid) with vehicle from Day 1 to Day 28 of the study.

Body weight was monitored twice per week and on Day 28. After sacrifice, blood glucose, body weight, body mass, serum MCP-1 and insulin levels were also recorded.

MCP-1 was measured by ELISA. The statistical analysis for p value was performed using the program Prism (Graphpad, Monrovia, Calif.) with the Student t-test (unpaired). All data are presented as the mean ± standard error of the mean (SEM).

As illustrated in this example, (S)-{[4-(1H-indol-3-yl)-piperidin-1-yl]}-{1-[(2E)-3-(3,4,5-trifluoro-phenyl)-acryloyl]-piperidin-4-yl}-acetic acid both significantly reduced weight gain and body fat mass gain with no change in lean mass.

Accordingly, said compound may be useful in preventing, treating or ameliorating obesity.

TABLE 1

| | Plasma | Body | | Change in | Change in | |
|---|---|---|---|---|---|---|
| | MCP-1 | Weight | Food Intake | Fat Mass | Lean Mass | Change in |
| Group | (pg/ml) | Gain (g) | (g/7 days) | (g) | (g) | Water (g) |
| TA1 Vehicle | 46.4 ± 3.4 | 5.3 ± 1.5 | 12.8 ± 4.7 | 1.8 ± 0.8 | 3.5 ± 0.7 | 3.2 ± 0.6 |
| TA1 25 mg/kg | 69.2 ± 5.6 | 3.8 ± 1.2 | 14.0 ± 1.6 | 0.9 ± 0.7 | 3.1 ± 0.9 | 2.8 ± 0.8 |
| TA1 50 mg/kg | 98.3 ± 7.6 | 3.4 ± 1.1* | 13.5 ± 1.8 | 0.4 ± 0.6 | 4.9 ± 0.9 | 4.5 ± 0.8 |
| TA1 100 mg/kg | 503.5 ± 69.7* | 2.3 ± 1.8* | 14.5 ± 3.6 | 0.2 ± 0.3* | 3.4 ± 0.3 | 2.9 ± 0.3 |
| TA2 Vehicle | 79.9 ± 15.8 | 2.1 ± 1.5 | 25.4 ± 3.2 | 1.0 ± 0.3 | 2.1 ± 0.6 | 1.9 ± 0.6 |
| TA2 100 mg/kg | 646.8 ± 112.5* | 2.3 ± 1.5 | 22.8 ± 1.7 | 0.6 ± 0.3 | 3.4 ± 0.8 | 3.1 ± 0.7 |

*P < 0.05, compared with non-treated group (One-way ANOVA).

### EXAMPLE 2

### Insulin Sensitivity Model

In an insulin sensitivity model in mice, four month old male NON (NONcNZO10/LtJ) mice in four treatment groups (treated: 25 mg/kg, 50 mg/kg and 100 mg/kg and non-treated vehicle control) were fed LabDiet® 5K20 for 28 days. The treated groups were dosed (0.1 mL, ip, bid) with a test compound from Day 1 to Day 28. The non-treated group was dosed (0.1 mL, ip, q.d.) with vehicle from Day 1 to Day 28.

Body weight and blood glucose were monitored once per week and an insulin tolerance test was performed by i.p. injection of 1.0 pL/kg insulin, then fed blood glucose levels were measured at 0, 15, 30, 60, 90, and 120 min after insulin administration. The insulin tolerance test blood glucose levels at the time points the sample were taken after insulin administration are shown in Table 5.

After sacrifice on Day 28, blood samples were collected to measure HbA1c, plasma levels of glucose, plasma insulin, FFA (free fatty acids) and MCP-1. The results of the Day 28 blood samples are shown in Table 6.

Blood glucose levels were measured using a Glucometer. MCP-1 and insulin were measured by ELISA. Plasma glucose and FFA were measured using Weko kits. The statistical analysis for p value was performed using the program Prism (Graphpad, Monrovia, Calif.) with the Student t-test (unpaired). All data are presented as the mean ± standard error of the mean (SEM).

As illustrated in this example, (S)-{[4-(1H-indol-3-yl)-piperidin-1-yl]}-{1-[(2E)-3-(3,4,5-triflouro-phenyl)-acryloyl]-piperidin-4-yl}-acetic acid increased insulin sensitivity, improved blood glucose control and significantly reduced the HbA1c levels without a significant difference in body weight between treated and non-treated groups.

Accordingly, said compound may be useful in a method for treating or ameliorating Type II diabetes and associated diabetic complications.

TABLE 2a

| | Blood Glucose (mg/dl) Day 7 | | | | | | |
|---|---|---|---|---|---|---|---|
| | 0 min | 15 min | 30 min | 60 min | 90 min | 120 min | p value |
| Vehicle | 326 ± 55 | 188 ± 15 | 163 ± 19 | 154 ± 15 | 148 ± 15 | 164 ± 23 | |
| 25 mg/kg | 333 ± 44 | 213 ± 40 | 157 ± 27 | 146 ± 21 | 146 ± 18 | 164 ± 23 | P > 0.05 |
| 50 mg/kg | 325 ± 54 | 213 ± 51 | 136 ± 20 | 115 ± 13 | 115 ± 8 | 142 ± 8 | P < 0.05 |
| 100 mg/kg | 230 ± 22 | 141 ± 16 | 105 ± 9 | 82 ± 8 | 83 ± 7 | 98 ± 8 | P < 0.01 |

TABLE 2b

| | Blood Glucose (mg/dl) Day 21 | | | | | | |
|---|---|---|---|---|---|---|---|
| | 0 min | 15 min | 30 min | 60 min | 90 min | 120 min | p value |
| Vehicle | 476 ± 60 | 384 ± 55 | 255 ± 40 | 199 ± 22 | 203 ± 28 | 216 ± 29 | |
| 25 mg/kg | 465 ± 34 | 367 ± 51 | 247 ± 53 | 209 ± 56 | 198 ± 51 | 237 ± 43 | P > 0.05 |
| 50 mg/kg | 434 ± 71 | 303 ± 56 | 186 ± 32 | 162 ± 37 | 168 ± 47 | 214 ± 49 | P < 0.05 |
| 100 mg/kg | 332 ± 38 | 247 ± 28 | 152 ± 15 | 144 ± 21 | 158 ± 28 | 228 ± 43 | P < 0.01 |

TABLE 3

| | Plasma | Plasma | Blood | Plasma | Plasma | Body |
|---|---|---|---|---|---|---|
| | MCP-1 | Insulin | HbA1c | Glucose | FFA | Weight |
| | (pg/ml) | (ng/ml) | (%) | (mg/dl) | (mM) | (g) |
| Vehicle | 67.2 ± 8.9 | 13.4 ± 2.0 | 7.3 ± 0.6 | 465 ± 80 | 1.3 ± 0.1 | 43.9 ± 1.6 |
| 25 mg/kg | 56.6 ± 3.0 | 5.8 ± 0.7* | 6.6 ± 0.5 | 413 ± 52 | 1.2 ± 0.4 | 45.2 ± 1.4 |
| 50 mg/kg | 177.4 ± 63.4 | 5.4 ± 0.9* | 6.7 ± 0.7 | 358 ± 58 | 1.0 ± 0.2 | 42.6 ± 0.8 |
| 100 mg/kg | 555.6 ± 104.5* | 7.9 ± 1.1* | 5.0 ± 0.2* | 190 ± 15* | 0.6 ± 0.1* | 43.6 ± 0.9 |

*P < 0.05, compared with non-treated group (One-way ANOVA)

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method for treating a subject having a syndrome, disorder or disease related to CCR2 activation, said method comprising administering to said subject an effective amount of a compound of Formula (I): 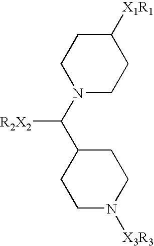
wherein
X₁ is absent, alkyl, carbonyl, alkylcarbamoyl or alkylcarbamoylalkyl,
R₁ is aryl or heterocyclyl, wherein heterocyclyl has an optionally present nitrogen atom and wherein the nitrogen atom is optionally oxidized, and wherein aryl and heterocyclyl are each optionally substituted with one or more of alkyl, alkoxy, cyano, halogen, hydroxy, hydroxyalkyl, nitro, amino (optionally substituted with one or more of alkyl, acyl, carbonylalkoxy, sulfonylalkyl, alkylcarboxy or alkylcarbonylalkoxy), alkylcarboxy, alkylcarbonylalkoxy, alkoxycarboxy, alkoxycarbonylalkoxy, alkylamino, alkylaminoalkyl, sulfonylamino, sulfonylaminoalkyl, alkylsulfonylamino, alkylsulfonylaminoalkyl, carboxy, acyl, carbonylalkoxy, carbamoyl or carbamoylalkyl,
X₂ is absent or alkyl,
R₂ is hydroxy, halogen, amino (optionally substituted with one or more of alkyl, formyl, acyl, sulfonylalkyl or carbonylalkoxy), cyano, nitro, alkoxy, carboxy, carbonylalkoxy, oxyacyl, oxyacylaryl, oxyacrylyl, oxyacrylylaryl (optionally substituted on aryl with one or more of alkyl, alkoxy, cyano, halogen, hydroxy, nitro, amino or aminoalkyl), oxycarbonylalkoxy, aminoacylamino, aminoacylaminoalkyl, carbamoyl, carbamoylalkyl, urea or ureaalkyl,
X₃ is carbonyl, carboxyl, acyl, acyloxy, acrylyl, carbonylalkynyl, carbonylalkoxy, carbamoyl, carbamoylalkyl, alkylcarbamoyl, thiocarbamyl or iminomethylaminocarbonyl, wherein when X₃ is carbonylalkoxy, then R₃ is optionally present, and
R₃ is cycloalkyl, aryl or heterocyclyl each optionally substituted with one or more of alkyl, alkoxy, cyano, halogen, alkyltrihalo, alkoxytrihalo, hydroxy, nitro, amino, aminoalkyl, alkylamino, alkylaminoalkyl, thioalkyl, thioalkyltrihalo, carboxy, acyl, carbonylalkoxy, carbamoyl, carbamoylalkyl or aryl (optionally substituted on aryl with one or more of alkyl, alkoxy, halogen, hydroxy, nitro, amino or aminoalkyl).

2. The method of claim 1, wherein the compound of claim 1 is (S)-{[4-(1H-indol-3-yl)-piperidin-1-yl]}-{1-[(2E)-3-(3,4,5-triflouro-phenyl)-acryloyl]-piperidin-4-yl}-acetic acid or a salt, isomer, prodrug, metabolite or polymorph thereof.

3. The method of claim 3, wherein the compound of claim 1 is (S)-{[4-(1H-indol-3-yl)-piperidin-1-yl]}-{1-[(2E)-3-(3,4,5-trifluoro-phenyl)-acryloyl]-piperidin-4-yl}-acetic acid or a salt, isomer, prodrug, metabolite or polymorph thereof.

4. The method of claim 3, wherein the compound of claim 1 is (S)-{[4-(1H-indol-3-yl)-piperidin-1-yl]}-{1-[(2E)-3-(3,4,5-trifluoro-phenyl)-acryloyl]piperidin-4-yl}-acetic acid or a salt, isomer, prodrug, metabolite or polymorph thereof.

5. The method of claim 3, wherein the syndrome, disorder or disease is selected from ophthalmic disorders, uveitis, atherosclerosis, rheumatoid arthritis, psoriasis, psoriatic arthritis, atopic dermatitis, multiple sclerosis, Crohn's Disease, ulcerative colitis, nephritis, organ allograft rejection, fibroid lung, renal insufficiency, Type II diabetes and diabetic complications, diabetic nephropathy, diabetic retinopathy, diabetic retinitis, diabetic microangiopathy, obesity, tuberculosis, chronic obstructive pulmonary disease, sarcoidosis, invasive staphyloccocia, inflammation after cataract surgery, allergic rhinitis, allergic conjunctivitis, chronic urticaria, asthma, allergic asthma, periodontal diseases, periodonitis, gingivitis, gum disease, diastolic cardiomyopathies, cardiac infarction, myocarditis, chronic heart failure, angiostenosis, restenosis, reperfusion disorders, glomerulonephritis, solid tumors and cancers, chronic lymphocytic leukemia, chronic myelocytic leukemia, multiple myeloma, malignant myeloma, Hodgkin's disease, and carcinomas of the bladder, breast, cervix, colon, lung, prostate, or stomach.

6. The method of claim 3, wherein the effective amount is from about 0.1 ng/kg/day to about 300 mg/kg/day.

7. The method of claim 5, wherein the syndrome, disorder or disease is obesity.

8. The method of claim 3, wherein the effective amount reduces weight gain and body fat mass gain.

9. The method of claim 7, wherein the syndrome, disorder or disease is selected from Type II diabetes and diabetic complications.

10. The method of claim 9, wherein diabetic complications are selected from diabetic nephropathy, diabetic retinopathy, diabetic retinitis or diabetic microangiopathy.

11. The method of claim 10, wherein the effective amount increases insulin sensitivity.

12. The method of claim 5, wherein the compound of claim 1 is administered in combination with one or more other therapeutic agents.

13. The method of claim 12, wherein the other therapeutic agent is one or more anti-inflammatory agents is selected from an antibiotic, corticosteroid or steroid anti-infective agent or immunosuppressive agent.
